(19) United States
(12) Patent Application Publication
(10) Pub. No.: US 2008/0033404 A1
(43) Pub. Date: Feb. 7, 2008

(54) Surgical machine with removable display

(76) Inventors: Laszlo O. Romoda, San Clemente, CA (US); Christopher Horvath, Irvine, CA (US)

Correspondence Address: ALCON, IP LEGAL, TB4-8, 6201 SOUTH FREEWAY, FORT WORTH, TX, 76134, US

(21) Appl. No.: 11/498,401

(22) Filed: Aug. 3, 2006

(51) Int. Cl. A61B 17/00

(52) U.S. Cl. 606/1

(57) ABSTRACT

A surgical system includes a main surgical unit, a display in communication with the main surgical unit, and a cradle for receiving the display when the display is removed from the main surgical unit. The display is removable from the main surgical unit so that the display can be located inside a sterile field of an operating room and the main surgical unit can be located outside the sterile field of the operating room.

### FIELD OF THE INVENTION

The present invention relates to surgical machines and more particularly to a surgical machine with a removable display that allows the integrity of a sterile field in an operating room to be preserved.

### BACKGROUND OF THE INVENTION

The need for a sterile space in an Operating Room (OR) is well known. In a typical OR, an area around the patient must remain sterile. This area is referred to as the sterile field. It is important that any surgical equipment placed in the sterile field remain sterile for the duration of the operation. The area outside the sterile field contains items, including surgical equipment, that do not need to be sterilized.

Many surgeries involve large pieces of surgical equipment. This equipment, or parts of it, must be sterilized if it is located in the sterile field. Most typically, equipment is draped with sterile plastic if it is to reside in the sterile field. Alternatively, a portion of the equipment, like a surgical hand piece, is located in the sterile field while the remainder of the equipment is located outside the sterile field. A remote control may then be provided to operate the piece of equipment that is not located in the sterile field.

Most electronic surgical machines have a user interface that allows the doctor to control the machine. These user interfaces may incorporate the latest in touch screen display technology. Using a touch screen display allows the doctor to receive information about how the machine is performing. A touch screen display can also receive instructions from the doctor in an easy and straightforward fashion. The user interface provided by a touch screen display can be intuitive and robust, providing the doctor with an effective way to perform surgery.

More particularly, ophthalmic surgery often involves complex surgical equipment in the form of a console with associated work pieces. This equipment is usually computer controlled and very sophisticated. A touch screen display is often employed to provide the doctor with important information about how the equipment functions. This same touch screen display may also receive input from the doctor to control the equipment during surgery. Accordingly, the equipment must be located close to the doctor so that he can see the display and input the proper control commands.

FIG. 1 is an example of a conventional surgical machine. Surgical machine 100 has a main surgical console 105 and an integral display 110. In this configuration, the integral display 110 cannot be removed from the main surgical console 105. When used in an operation, the entire surgical machine 100 must be placed outside the sterile field in order to maintain sanitary conditions in the OR. Alternatively, the surgical machine 100 can be enclosed with sterile plastic so that it can be placed in the sterile field. In this manner, the sterile field can remain sterile, and the surgeon can access the surgical machine 100.

Since the surgical machine is located in the OR, maintaining a sterile environment is important. It can be cumbersome to drape the entire surgical machine so that it can be reside in the sterile field. However, this may be necessary so that the machine controls are close to the doctor. Another alternative is to provide the doctor with a remote control device. This remote control device can be used to direct the operation of the machine while the machine is located outside of the sterile field. However, these remote control devices may not be as robust as a full-featured touch screen display. It would be desirable to locate the touch screen display in the sterile field while the remainder of the equipment is located outside the sterile field.

### SUMMARY OF THE INVENTION

In one embodiment consistent with the principles of the present invention, the present invention is a surgical system having a main surgical unit and a display in communication with the main surgical unit. The display is removable from the main surgical unit so that the display can be located inside a sterile field of an operating room and the main surgical unit can be located outside the sterile field of the operating room.

In another embodiment consistent with the principles of the present invention, the present invention is a surgical system having a main surgical unit, a display in communication with the main surgical unit, and a cradle for receiving the display when the display is removed from the main surgical unit. The display is removable from the main surgical unit so that the display can be located inside a sterile field of an operating room and the main surgical unit can be located outside the sterile field of the operating room.

In another embodiment consistent with the principles of the present invention, the present invention is a surgical system having a main surgical unit, a touch screen display, and a cradle. The main surgical unit is capable of performing a surgical procedure and has a first connector for receiving a first signal. The touch screen display is removable from the main surgical unit and is adapted to couple with the first connector located on the main surgical unit. When the display is coupled to the first connector, it can transmit a signal to the main surgical unit via the first connector. The cradle can receive the touch screen display when the touch screen display is removed from the main surgical unit. The cradle also has a second connector for receiving a second signal. When the touch screen display is coupled to the cradle via the second connector, the touch screen display can transmit a signal to the cradle through the second connector. The cradle can transmit this signal to the main surgical unit. The touch screen display is removable from the main surgical unit so that the touch screen display and cradle can be located inside a sterile field of an operating room and the main surgical unit can be located outside the sterile field of the operating room.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a conventional surgical machine.

FIG. 2 is a perspective view of a surgical machine with a removable display according to an embodiment of the present invention.

FIG. 3 is a perspective view of a surgical machine with a removable display and a receiving cradle according to an embodiment of the present invention.

FIG. 4 is a perspective view of a surgical machine with a removable display and a receiving cradle according to an embodiment of the present invention.

FIG. 5 is an illustration of a surgical machine with a removable display as used in an OR according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

FIG. 2 is a perspective view of a surgical machine with a removable display according to an embodiment of the present invention. Surgical machine 200 comprises a main surgical unit 205 and a removable display 210. In this configuration, the removable display 210 can be removed from the main surgical unit 205. The removable display 210 matingly couples with the main surgical unit 205 via main connector 215. Main connector 215 allows the removable display 210 to communicate with the main surgical unit 205 when they are coupled together. Main connector 215 allows signals to pass between the main surgical unit 205 and the removable display 210 when the removable display 210 is connected to the main surgical unit 205.

Removable display 210 conveys information about the surgical machine to a doctor. For example, in ophthalmic surgery, removable display 210 displays information about the how a cataract or retinal surgery machine is operating. Removable display 210 typically shows functional parameters such as temperature or fluid pressure. Removable display might also show various steps that are performed during an ophthalmic procedure.

In one embodiment of the present invention, removable display 210 is a touch screen display. Touch screen displays allow a user to input a command by touching the screen of the display. For example, a doctor can start a procedure by touching a “start” icon displayed on the screen of the removable display 210. In this manner, the removable display 210 receives a control input (that the doctor wishes to start the procedure) by sensing the pressure of the doctor's finger on the screen of the removable display 210. The surgical machine 200 can then respond to the control input.

In other embodiments of the present invention, removable display 210 is a liquid crystal display (LCD), plasma display, or other flat panel display. In this case, the removable display can provide information to the doctor about the function of the surgical machine or the status of operation itself. For example, the removable display 210 may display information about the completion of a certain step in the operation.

In one embodiment of the present invention, main surgical unit 205 contains a computer, control circuitry, and other mechanical devices (not shown) used to perform an operative procedure. Other surgical devices, such as operating hand pieces, can be connected to the front panel 220 of main surgical unit 205. The main surgical unit 205 houses the computer and control circuitry that send information to the removable display 210. In this manner, the main surgical unit 205 controls the information that is displayed on the removable display 210. The computer and control circuitry housed in the main surgical unit 205 also responds to any commands entered into the removable display 210 when the removable display 210 is a touch screen display.

FIG. 3 shows an embodiment of the present invention which utilizes a cradle 300 for receiving the removable display 210. In this embodiment, removable display 210 is removed from the main surgical unit 205 and placed in cradle 300. Cradle 300 is adapted to matingly connect with removable display 210 via cradle connector 305. Like main connector 215, cradle connector 305 allows the removable display 210 to communicate with the main surgical unit 205. Cradle connector 215 allows signals to pass between the main surgical unit 205 and the removable display 210 when the removable display 210 is connected to cradle 300. Cradle 300 may also have control or interface features on its front face 310.

In one embodiment of the present invention, the cradle 300 is connected to the main surgical unit 205 via cable 315. Cable 315 allows signals to pass between the cradle 300 and the main surgical unit 205. In this manner, the main surgical unit 205 can send information to be displayed on removable display 210 via cable 315, cradle 300, and cradle connector 305. For example, information about the temperature of a surgical hand piece can be sent from main surgical unit 205, through cable 315, to cradle 300, through cradle connector 305, and to the removable display 210. Likewise, a control input entered by a doctor when removable display 210 is a touch screen display proceeds as follows: from removable display 210, through cradle connector 305, to cradle 310, through cable 315, to main surgical unit 205.

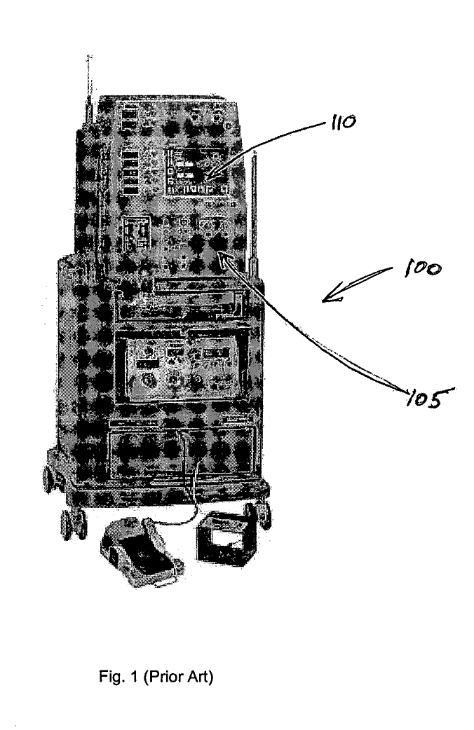

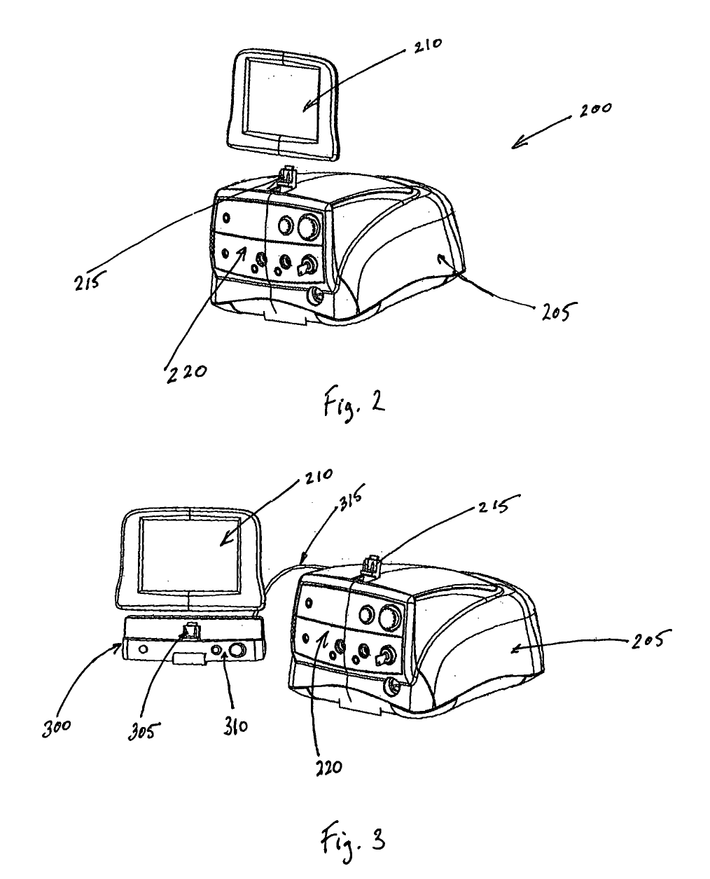

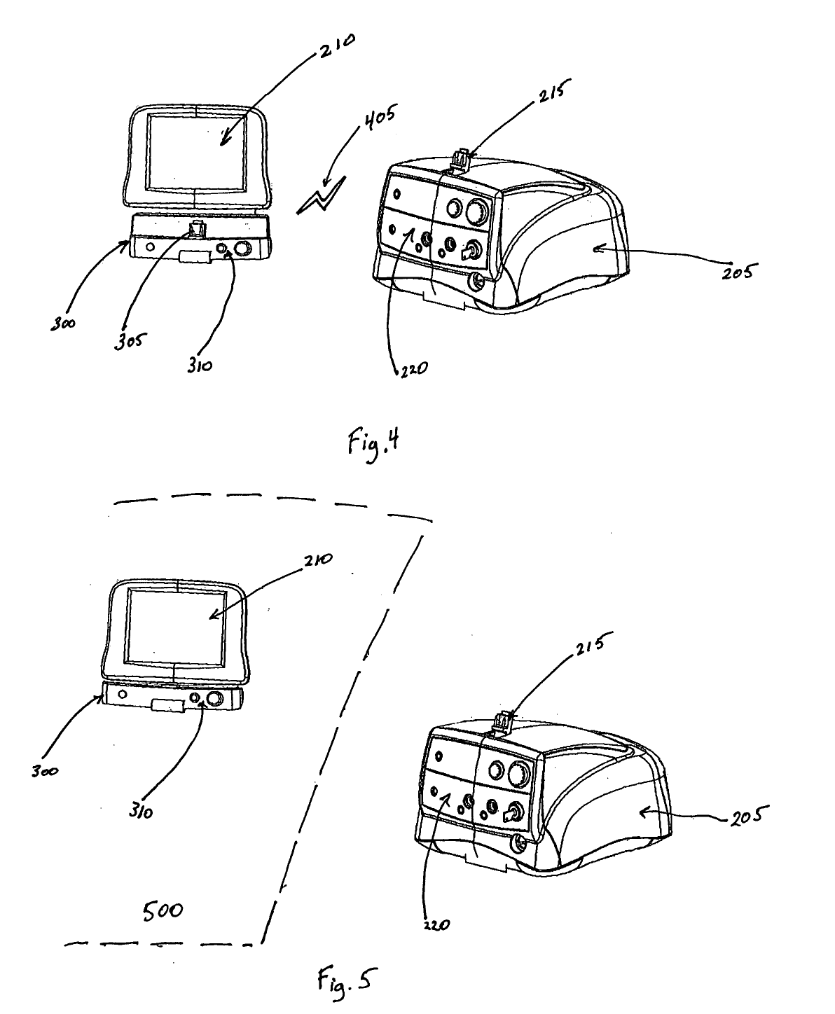

In another embodiment of the present invention shown in FIG. 4, cable 315 is not present. In this configuration, a wireless connection 405 is established between main surgical unit 205 and cradle 300. For example, both main surgical unit 205 and cradle 300 may be Bluetooth enabled. In this manner, signals pass through the air between cradle 300 and main surgical unit 205 utilizing a Bluetooth protocol. Main surgical unit 205 may also communicate with cradle 300 using infrared (IR) signals or any other wireless protocol. Care should be taken to select a wireless communication protocol that does not interfere with other equipment in the OR.

With respect to FIGS. 3 and 4, the communication functionality of cradle 300 may be incorporated into removable display 210. In this configuration, cradle 300 is not present. Instead, removable display 210 contains all the functionality necessary to communicate with main surgical unit 205 when removable display 210 is removed from main surgical unit 205. For example, removable display 210 can be removed from main surgical unit 205 and placed on a table. Removable display 210 and main surgical unit 205 can then communicate via a cable 315 or wirelessly as described above.

FIG. 5 shows main surgical unit 205, removable display 210, and optional cradle 300 in an OR environment. Removable display 210 is nested in cradle 300. Both the display 210 and the cradle 300 are in the sterile field 500. Main surgical unit 205 is not in the sterile field 500. To maintain the integrity of the sterile field in an OR, anything that enters the sterile field must be sterile. Metal implements, such as forceps and scalpels, can be sterilized using an autoclave before being placed in the sterile field. However, a surgical machine, like one used for cataract or retinal surgery, would be difficult to sterilize. Instead, it is usually draped with sterile plastic and moved into the sterile field.

In FIG. 5, the removable display 210 and optional cradle 300 can be draped with plastic to maintain the integrity of the sterile field 500. This allows the surgeon to see the information displayed on removable display 210 and to interact with removable display 210 when it is a touch screen display. Since the removable display 210 is in the sterile field 500 where the surgeon is operating, it is much easier for him to interact with it. In addition, only the removable display 210 and optional cradle 300 need to be draped with plastic. The main surgical unit 205 can remain undraped and outside of the sterile field.

From the above, it may be appreciated that the present invention provides an improved machine and methods for maintaining the integrity of the sterile field in an OR. The present invention allows a surgeon to conveniently utilize the full functionality of a surgical machine without having to place the entire machine in the sterile field. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A surgical system comprising:
a main surgical unit; and
a display in communication with the main surgical unit;
wherein the display is removable from the main surgical unit so that the display can be located inside a sterile field of an operating room and the main surgical unit can be located outside the sterile field of the operating room.

2. The system of claim 1 wherein the display is in wireless communication with the main surgical unit.

3. The system of claim 1 wherein the display communicates with the main surgical unit via a cable.

4. The system of claim 1 wherein the display communicates with the main surgical unit via a Bluetooth signal.

5. The system of claim 1 wherein the display communicates with the main surgical unit via an infrared signal.

6. The system of claim 1 wherein the display is a touch screen display.

7. The system of claim 1 wherein the main surgical unit further comprises a first connector adapted to couple with the display.

8. A surgical system comprising:
a main surgical unit;
a removable display; and
a cradle for receiving the display when the display is removed from the main surgical unit.
wherein the display is removable from the main surgical unit so that the display and the cradle can be located inside a sterile field of an operating room and the main surgical unit can be located outside the sterile field of the operating room.

9. The system of claim 8 wherein the cradle communicates wirelessly with the main surgical unit.

10. The system of claim 8 wherein the cradle communicates with the main surgical unit via a cable.

11. The system of claim 8 wherein the cradle communicates with the main surgical unit via a Bluetooth signal.

12. The system of claim 8 wherein the cradle communicates with the main surgical unit via an infrared signal.

13. The system of claim 8 wherein the display is a touch screen display.

14. The system of claim 8 wherein the main surgical unit further comprises a first connector adapted to couple with the display and the cradle further comprises a second connector adapted to couple with the display.

15. A surgical system comprising:
a main surgical unit with a first connector for receiving a first signal, the main surgical unit adapted to perform a surgical procedure;
a touch screen display removable from the main surgical unit, the touch screen display adapted to couple with the first connector, and when coupled with the first connector, the touch screen display transmitting the first signal to the main surgical unit via the first connector; and
a cradle for receiving the touch screen display when the touch screen display is removed from the main surgical unit, the cradle having a second connector for receiving a second signal, and when the touch screen display is coupled to the cradle via the second connector, the touch screen display transmitting the second signal to the cradle through the second connector and the cradle transmitting the second signal to the main surgical unit;
wherein the touch screen display is removable from the main surgical unit so that the touch screen display and cradle can be located inside a sterile field of an operating room and the main surgical unit can be located outside the sterile field of the operating room.

16. The system of claim 15 wherein the cradle communicates wirelessly with the main surgical unit.
